(19) United States
(12) Patent Application Publication
(10) Pub. No.: US 2002/0099356 A1
(43) Pub. Date: Jul. 25, 2002

(54) Transmembrane transport apparatus and method

(76) Inventors: Evan C. Unger, Tucson, AZ (US); Yunqiu Wu, Santa Clara, CA (US)

Correspondence Address: Gavin J. Milczarek-Desai, Durando Birdwell & Janke, P.L.C., 2929 E. Broadway Blvd., Tucson, AZ, 85716, US

(21) Appl. No.: 09/766,284

(22) Filed: Jan. 19, 2001

(51) Int. Cl. A61N 1/30; A61B 17/20; A61M 37/00; A61M 31/00; A61B 5/00; B65D 81/00; A61B 19/00

(52) U.S. Cl. 604/501; 128/898; 604/20; 604/22; 604/46; 600/575; 600/576

(57) ABSTRACT

A drug delivery device and method comprising first creating channels or pores across a biological membrane and secondly creating a driving force to propel drugs across or withdraw biological fluids through the membrane.

### BACKGROUND OF THE INVENTION

1. Field of Invention

This invention generally relates to the field of drug delivery systems and in particular to a device and method that utilize sonic and electric energy to deliver drugs or sample bodily fluids through the skin.

2. Description of the Related Art

The treatment of illness often requires the delivery of medicinal drugs to a patient's bloodstream (for systemic effect) or to a particular site of pathogenesis, such as a localized tumor. In either case, this traditionally has been accomplished in one of three ways: through oral administration (e.g. a pill), injection, or by adsorption through the skin. While all three drug delivery routes can be used efficaciously, each has drawbacks. The primary drawback to introducing a drug orally is that it undergoes gastrointestinal metabolism. The deleterious effects of metabolism can include drug degradation, exposure of, and damage to, the liver, and digestive maladies such as upset stomach or acid reflux. Furthermore, it may be wasteful or otherwise undesirable to treat a local condition with the system-wide delivery of a drug. Thus, there has been an ongoing need for alternatives to oral administration of a drug.

Transdermal (i.e. “across or through the skin”) methods deliver drugs without involving the digestive system. The two basic forms of transdermal delivery include the injection of a drug through a syringe and needle and topical application. Besides the pain associated with injections, drawbacks include the chance of infection and the problem of having drugs cleared from the blood too quickly, requiring subsequent injections to maintain effective concentrations.

For topical administration, the main drawback is that drug penetration is hampered by the relatively low permeability of skin. This is because the barrier properties of the skin allow only for the passage of small, uncharged or polar molecules, such as diatomic oxygen, glycerol, or water. Accordingly, polar molecules larger than water and charged molecules, such as certain amino acids or hydrogen ions, generally do not diffuse across the skin. See Cooper, G. M., The Cell: A Molecular Approach. Chapter 2 “The Chemistry of Cells,” p. 81, ASM Press, Washington D.C. (1997). Thus, therapeutically relevant rates of drug delivery often are difficult to achieve by applying a drug to the surface of the body because typical drugs are too large and/or charged to readily diffuse through the skin.

One approach to overcoming the barrier properties of the skin entails the use of delivery vehicles containing penetration enhancing compounds, such as surfactants, lipids and other aliphatic compounds, liposomes and niosomes. While these compounds increase drug absorption through the skin to some extent, problems with developing pharmaceutically acceptable, stable formulations of both the delivery vehicle, and the drug harbored within, persist.

Another improved transdermal delivery method involves the use of microneedles. In contrast to traditional injections with standard-gauge hypodermic needles, microneedle (radius of curvature ˜1 um) arrays permeabilize the skin by producing microscopic holes through the stratum corneum (SC) layer of skin. These holes, in effect, act as conduits for drug delivery. Due to their small size and the fact that they are fabricated to penetrate approximately the first 150 um of the skin (well above sensory nerves), microneedle arrays can accomplish transdermal delivery without significant pain. See Henry, S. et al., Microfabricated Microneedles: A Novel Approach to Transdermal Drug Delivery. J. of Pharmaceutical Sciences, 7(8):922-925 (August, 1998). However, while the use of microneedle arrays increases the amount of drug diffusion through the skin and into the bloodstream, it does not actively promote the local cellular uptake of the delivered drug.

Further improvements in transdermal drug delivery techniques involve the use of electricity. It is well known that electricity augments transdermal delivery, a process called electro- or iontophoresis. It is also known that applying a strong electric field to cells can cause a phenomenon known as electroporation, which actively promotes uptake of drugs through transient cell membrane disruptions caused by the electric field.

Using iontophoresis, penetration through the skin of ionic drugs can be optimized by using an applied voltage whereby the electrical energy increases the local concentration of the medication at the desired site. Thus, iontophoresis of a charged drug is thought to be accomplished due to simple ion interactions during which the charged drug is repelled from a like-charged portion of the delivery system and attracted to the oppositely-charged portion located at the target for injection. Gangarosa, L. P. Sr. and James M. Hill, Modern Iontophoresis for Local Drug Delivery. International Journal of Pharmaceutics 123, pp. 159-171 (1995). However, this technique is not useful with uncharged drugs.

In lieu of delivering a drug across the skin and into the bloodstream, it is sometimes desirable to deliver a drug across the skin and to particular cells. Such a situation may occur, for example, when administering chemotherapeutic agents or genetic materials to localized tumor cells. In this case, the main goal of drug delivery is to promote the uptake of the drug by the diseased cells while minimizing uptake by surrounding tissue or the blood. Electroporation is now commonly used in vitro and in vivo to achieve this goal. Electroporation is thought to work by transiently making cell membranes more permeable by the action of short electric impulses, which, in effect, open a “pore” in the membrane structure through which a drug may pass into the cell's cytoplasm. See Neumann, E., et al., Gene Transfer Into Mouse Lyoma Cells By Electroporation In High Electric Fields. EMBO Journal 1(7):841-845 (1982).

Electroporation technology has recently been adapted for use in human patients. For example, U.S. Pat. No. 5,810,762 by Hofmann describes an improved electroporation system that assists a user in properly locating a pair of spaced electrodes such that the application of an electric field promotes cellular uptake of a drug in a localized area of the body. However, drugs must still be infused or otherwise delivered into the target site before electroporation can be performed, raising the same drawbacks discussed above.

Combining needles with electricity to penetrate the skin and deliver a drug intracellularly is also known as described in published patent application WO97/07826 by Nicolau et al. This patent application describes a method of treating mammalian cells in vivo by, for example, separately injecting nucleic acids and using an array of electrically conductive needles at the site of injection to induce electroporation of the nearby cells.

Similarly, published patent application WO00/05339 by Canham discloses a method and apparatus for electroporating cells by growing the cells on microneedles of conductive silicon and subjecting the silicon to an electric field.

Another approach aimed at both improved transdermal delivery and enhanced cellular uptake has utilized sound waves to aid in the permeabilization of barriers to transdermal drug delivery. For example, researchers have employed the use of a device that delivers drugs across the main barrier to drug delivery, the stratum corneum (SC) layer of the skin, by pulsing the skin with low-frequency (˜20 khz) sound waves. This treatment of the skin with sound waves induces a phenomenon known as “cavitation” in the cell membranes of the dead, keratin-filled cells composing the SC. Mitragotri, S. et al, Ultrasound-Mediated Transdermal Protein Delivery. Science 269:850-853 (1995).

However, cavitational effects are inversely proportional to ultrasound frequency. Accordingly, only molecules with a molecular weight of less than 6,000 can be transported across the skin at therapeutic frequencies in the 1 MHz range. To overcome this problem, lower frequency sound waves (e.g. 20 MHz) have been used. However, the ultrasonic waves must be applied for 1 or more hours before obtaining substantial transport of the molecules that were tested on human skin. (Mitragotri). Moreover, the usefulness of this approach to deliver other macromolecules is presently unknown. Thus, this method has the disadvantages of inconvenience to the patient and unknown reliability with the wide assortment of available drugs.

Another example of using sound waves to assist in drug delivery is U.S. Pat. No. 5,733,572, issued to Unger et al., which provides for gas-filled microspheres as topical and subcutaneous delivery vehicles. These microspheres are made to encapsulate drugs and are injected or otherwise administered to a patient. Ultrasonic energy is then used to rupture the microspheres so as to release the drug at an appropriate target. Application of ultrasound for this purpose typically involves production of ultrasound at frequencies between 0.5 MHz and 10 MHz. As discussed above, this range of frequencies have been shown to be of limited use in producing cavitation effects in skin cells, which are much larger than the size of typical microspheres.

To produce cavitation in the skin, relatively high levels of ultrasound energy may be required. This may increase the temperature of the skin and potentially cause burns. While the use of gas filled microspheres may lower the cavitation threshold and the amount of energy needed for transdermal transport, other mechanical methods are still needed to optimize transdermal drug delivery. Conventional systems may need larger power supplies to generate sufficient power to drive the drug across the skin. To make portable drug delivery devices that a patient can conveniently wear on the skin, it is useful to mechanically lower the resistance for transport across the skin. Accordingly, it may be possible to lower the energy requirements and use smaller and cheaper power supplies.

Yet another application of ultrasound or iontophoresis is to sample body fluids. By causing transient disruptions in cellular membranes, body fluids may be drawn through the skin. The result is that one may sample the concentration of biologically important compounds such as electrolytes, glucose, biomolecules and drugs. Rao, G., et al., Pharm. Res. (1995) 12:1869-1873. However, these two techniques still suffer from many potential drawbacks as described above, including molecule size and charge limitations, high power requirements, or inconveniently long duration of the extraction procedure.

Finally, it has been suggested in the related art that several methods of permeabilizing cells (i.e. mechanical, physical, and chemical means) be combined to enhance the introduction of topically applied drugs into skin or muscle cells (see in particular published application WO00/02621 at page 18. However, none of the related art is known to disclose or suggest a specific device or method utilizing sonic energy, or the combination of sonic energy and electric energy, to deliver drugs subdermally through a microneedle array such that local or systemic delivery, or sampling of biological fluids, may be achieved.

Therefore, there is a need for a device and method that is adapted to provide for improved delivery or local cellular uptake of a wide variety of drugs and for extraction and monitoring of biological fluids.

### SUMMARY OF THE INVENTION

The invention meets the aforementioned need by providing an improved drug delivery device and method. The invention encompasses a device and method of firstly creating pores through membranes, for example, the cells that form skin, and secondly creating a driving force to hasten passage of bioactive materials across such pores. In a preferred embodiment of the invention, a microneedle array device that utilizes sonic energy to deliver or extract biomolecules through membranes is provided.

As used herein and consistent with its commonly known meaning, the term “pores” defines minute openings in a biological membrane. Although the size of such openings may vary from about 5 microns up to 1000 microns, they typically will range from about 10 to 150 microns. As such, pores are intended to be distinguished from perforations or holes made in tissue by, for example, hypodermic needles or scalpels.

The preferred method of the invention generally includes a two-step process for biomolecule delivery or extraction: creation of pores across a membrane and enhanced deposit or withdrawal of a substance of interest. The membrane through which the pores are made may include a mucous membrane, such as inside the mouth, an intestinal surface, such as the stomach, small bowel, or colon, or the inside of an airway such as the trachea. The membrane may further include an endoluminal surface, such as the peritoneum, the surface of an organ (e.g. the heart or the kidney or the brain or spinal cord), or the inside of a blood vessel. For endovascular applications, the device may be fashioned upon a catheter and deployed with a balloon, e.g. an angioplasty balloon. Preferably the membrane is the skin. In general, the device is used by attaching it to the surface of the biological membrane such as the skin. For other membranes, the device may be attached to the surface of the membrane through surgical exposure, if necessary, or by using an endoscopic technique.

The inventive method also may be accomplished by abrading the surface of the membrane with an abrasive material such as a paper coated with coarse silicon granules (e.g. sandpaper) or by affixing a removable pad coated with a plurality of microneedles. In general, the needles are between about 15 microns to 5,000 microns long, with about 50 to 1,5000 microns in length more preferred and about 150 microns in length most preferred. The width of the needles may vary from about 5 microns to up to 1,000 microns, and most preferably from about 10 to 150 microns. The needles may be solid or contain a central bore through which fluid may pass. The needles may be shaped as cylindrical points or as serrated edges, e.g. as miniature daggers.

In the case of a removable pad bearing a plurality of microneedles, when the microneedles are removed after application to, for example, the skin, a series of pores are left upon the skin. The drug delivery device may then be affixed to the pre-permeabilized or pore-containing skin. Additional ways of creating the pores include burning holes in the skin, either with lasers or heated electrodes. Preferably, if holes are burned, the holes are about 15 to 5,000 microns deep into the skin and more preferably between about 50 and 1,000 microns deep, with between about 100 and about 300 microns deep being most preferable. The holes may be burned using a patch comprising a plurality of heating elements, e.g. microelectrodes.

Preferably, more than one pore is formed in the skin or the surface of the membrane across which drugs are to be delivered, and even more preferably over 10 pores. The number of pores may range from about 10 to about 100,000, but usually will be from about 50 to more than about 10,000. In general the number of pores will depend upon the size of the pores, the intended application, and the drug to be delivered. For example, larger molecular weight drugs at higher doses may require more and larger pores.

Yet another way of creating pores is to use a compressed gas. A nozzle from a cylinder of compressed gas may be affixed to the skin. By releasing the compressed gas, the gas accelerates and strikes the skin at high velocity, resulting in micropores and also introducing gas into the tissue. The microcavities of gas resulting in the tissue afford the added benefit of creating cavitation nuclei within the skin. This interacts with ultrasound (described below) to increase its effectiveness for transdermal transport.

To increase the poration effect on the skin or membrane by the gas, the nozzle may be designed to include a plurality of micronozzles, wherein the gas is directed in high speed streams to create a plurality of micropores in the skin. The diameter of these micronozzles may vary from about 5 microns to up to about 5 millimeters, and more usually between about 30 microns and about 2 millimeters. A wide variety of compressed gases may be used in this regard, including but not limited to air, nitrogen, carbon dioxide, argon, neon, helium, sulfur hexafluoride and pefluorocarbon and hydrofluorocarbon gases ranging from 1 to 6 carbons in size and from 4 to 22 hydrogen and/or fluorine atoms. In particular air, helium and fluorocarbon gases are preferred. The gas may include a mixture of more than one gas, e.g. a perfluorocarbon gas with air or helium. By selecting the proper gas and conditions, the invention can create pores of the proper size containing microbubbles. By adjusting the size of these microbubbles and pores, a resonance phenomenon may be obtained with the ultrasound to optimize transport of the drugs with ultrasound. Also electrically conducting gases such as xenon and hyperpolarized gases may be employed to improve electrical conductivity of the skin.

After creating the micropores in the skin, the invention comprises a driving force to improve transport of drug across the membrane. The driving force may comprise a pressure gradient, caused by a static pressure or a dynamic pressure wave as in ultrasound or the combination also of a static pressure source and a pressure wave. In general the reservoir of drug is affixed to the skin and the pressure is applied from behind the drug reservoir, with the surface of the drug reservoir including a semipermeable membrane affixed to the physiological membrane that has been pre-treated to create pores.

Pressure or a driving force can be created by ultrasound as described further below to generate a pulsatile form of pressure, i.e. using waves, but also by using a static pressure. The static pressure may be created in many ways, for example, by using a piston, from a liquid converting to a gas, or from a solid converting to a gas (e.g. bicarbonate crystals forming carbon dioxide gas) to create pressure upon the backing of the drug reservoir. If a piston is used the pressure may be generated from an actuator acting upon a piston to generate hydrostatic pressure within the drug reservoir or patch.

Furthermore, pressure may be created by a liquid gaseous precursor entrapped within the backing material behind the drug reservoir. As the liquid material converts to gas, it exerts pressure upon the drug reservoir, thereby increasing the driving pressure into the skin. In this regard, heating coils may be affixed to the back of the device to increase the head space of gas pressure exerting upon the backing of the drug reservoir. Accordingly, a circuit wherein the circuit is controlled can drive the heating coils from inputs. Inputs sensitive to biological indicators, e.g. drug concentration, can be used to activate the heating coils and increase pressure in response to a biological signal.

A variety of gaseous precursor materials may be employed in this invention, including isobutane and perfluoropentane as mentioned above. In fact, virtually any liquid can be used to make gaseous precursors so long as it is capable of undergoing a phase transition to the gas phase upon passing through the appropriate temperature. For example, suitable gaseous precursors for use in the present invention are the following: hexafluoroacetone, isopropyl acetyline, allene, tetrafluoro-allene, boron trifluoride, isobutane, 1,2-butadiene, 2,3-butadiene, 1,3-butadiene, 1,2,3-trichloro-2-fluoro-1,3-butadiene, butadiyne, 2-methyl-1,3-butadiene, hexafluoro-1,3-butadiene, 1-fluoro-butane, 2-methyl-butane, decafluorobutane, 1-butene, 2-butene, 2-methyl-1-butene, 3-methyl-1-butene, perfluoro-1-butene, perfluoro-2-butene, fluoroethane, nitropentafluoroethane, ethyl vinyl ether, trifluoromethanesulfonylchloride, trifluoromethanesulfonylfluoride, bromodifluoronitrosomethane, iodomethane, methyl ether, neon, neopentane, nitrogen (N₂), oxygen (O₂), n-pentane, perfluoropentane, 2-aminopropane, hexafluoropropane, trifluroacetonitrile, trifluoromethylperoxide, trifluoromethylsulfide, tungsten hexafluoride, vinyl acetylene, vinyl ether, and xenon. Most preferred are gasesous precursors that go from a liquid to a gas at a temperature around that of normal body temperature (37° C.). Such precursors include, but are not limited to, perfluoropentane, 1-fluorobutane, 2-methylbutane (isopentane), 2-methyl-1-butene, 2-methyl-2-butene, and 3-methyl-1-butyne.

Although the method of the invention may be accomplished with the use of more than one device, for example, pore creation with a microneedle patch followed by placement of an electrically conductive, drug-containing patch, more preferably the method is carried out by a single device with all required functions integrated into one unit. Most preferably, the device comprises a microneedle array in operable connection with a drug reservoir and an ultrasound transducer. Optionally, electrode elements are also included.

An object of this invention is to provide a combined sonic and microneedle arrangement that increases local cellular uptake of drugs at the area of injection.

A second object of this invention is to allow drug delivery to take place at therapeutic frequencies of ultrasound, providing a synergistic effect that both medicates and assists in healing the treatment area.

A third object of this invention is to decrease the amount of energy required to deliver a drug, thus achieving a higher more efficient delivery of medication at a lower power energy level.

A fourth object of the invention is to sample biological fluids and to analyze concentrations of biologically relevant materials in the biological fluids.

A fifth object is to provide pulsatile or effective bolus delivery of a pharmaceutical compound across the skin or other biological membrane.

A sixth object of the invention is to integrate monitoring of concentrations of biologically important compounds with delivery of therapeutic compounds such that therapeutic materials are then administered at the appropriate rate for best physiologic control.

Various other purposes and advantages of the invention will become clear from its description in the specification that follows and from the novel features particularly pointed out in the appended claims. Therefore, to the accomplishment of the objectives described above, this invention consists of the features hereinafter illustrated in the drawings, fully described in the detailed description of the preferred embodiments and particularly pointed out in the claims. However, such drawings and description disclose only some of the various ways in which the invention may be practiced.

### BRIEF DESCRIPTION OF THE DRAWINGS

FIG. 1 shows a schematic, cross-sectional view of an embodiment of the device of the invention.

FIG. 2 illustrates schematically a side view of a preferred embodiment of the device.

FIG. 3 shows a perspective view of one of the electrical insulating and conducting layers housed in the device of FIG. 2.

FIG. 4 shows a top view of the layers of fibers used to assemble the microneedles seen in FIG. 2.

FIG. 5A shows a perspective view of a series of the electrically conducting layers of FIG. 3.

FIG. 5B shows a close-up view of the microneedles housed in the conductive layers of FIG. 5A.

FIG. 6A shows a top view of the sheets of FIG. 5A being rolled to create concentric layers of conducting and insulating layers.

FIG. 6B shows a close-up view of the contents of the separate layers of FIG. 6A.

FIG. 7 shows a top view of the rolled layers of FIG. 6A after an end portion has been displaced to reveal a circular array of microneedles.

FIG. 8 shows a side cross-sectional view of the basic components of a preferred embodiment.

FIG. 9 shows a side view of the microarray needle and transducer assembly of a preferred embodiment.

### DETAILED DESCRIPTION OF THE INVENTION

The invention in general provides a drug delivery device that includes an ultrasonic transducer, a source of alternating current, a microneedle array and a biomolecule reservoir. The combined energy arrangement effectively decreases the required energy level, thus achieving a higher therapeutic index.

In one preferred embodiment, the device of the invention has a piezoelectric element that produces sound waves in response to an electric field. Since the microneedle elements lower the mechanical resistance across the skin, the ultrasound energy helps to drive the drug across the areas of lowered resistance, greatly increasing the rate at which drugs can be administered. By increasing the power applied to the transducer elements and increasing the rate of pulsing of ultrasound, the drug delivery rate may be increased. Similarly, these same techniques may be employed to draw biological fluids from the skin or other membranes.

The power supply to the transducer elements may be controlled by a pre-fixed program or modulated depending upon inputs from biological measurements. For insulin delivery, for example, measurement of glucose and/or hemoglobinA-1c may be utilized. A first device, which may be a transdermal measuring device employing ultrasound and a microneedle array, with or without iontophoretic elements measures, a biological indicator. Signals corresponding to the concentrations of the biological indicator(s) are then relayed to the second device, which comprises the delivery unit (needle microarray, drug reservoir, ultrasound and optional iontophoretic elements).

The energy driving the ultrasound and iontophoretic elements is then modulated depending upon the inputs from the measuring device. In the case of diabetes, the system allows prompt therapeutic delivery of insulin as needed to control the patient's blood sugar. While the microneedle array affords a low level of transdermal resistance, the microneedle array is insufficient to provide pulsatile, on-demand drug delivery and insufficient for rapid delivery of drugs. Thus, the application of energy to the piezoelectric and electrical elements affords great control over drug delivery rate.

In another preferred embodiment, the inventive device utilizes both electric current and sound waves to transport a drug through the skin and into the bloodstream or specific cells depending on the electric field strength and frequency of sound waves used. Ultrasonic frequencies between 50 kHz to 10 MHz may be employed, with frequencies between 0.5 MHz to 2 MHz being most preferred.

Materials for the acoustic element include piezoelectric materials such as ceramic materials including polymers, ceramics and micromachined silicon wafers. See Van Lintell, et al., Sensors and Actuators (1988) 15(2):153-167. Among the materials of the polymeric type are included PVDF (polyvinylidene fluorides) and PVDF-TRFE (polyvinylidene fluoridetrifluoroethylene). Chan, H. L. W., et al. (2000) IEE Transacts. On Dielectrics and Electrical Insulation, vol. 7(2) pp.204-207. Exemplary ceramics include lead zirconate-titanate (PZT) with or without dopants, lead titanate and metiniodates. The polymeric materials are especially useful for low power applications.

In another preferred embodiment, the inventive device utilizes both electric current and sound waves to transport a drug through the skin and into the bloodstream or specific cells depending on the electric field strength and frequency of sound waves used. Ultrasonic frequencies between 50 kHz to 10 MHz may be employed, with frequencies between 0.5 MHz to 2 MHz being most preferred. The power to the transducers is preferably via a battery source, although for patients within the hospital the power source may be from AC current but which may also be converted to DC. A battery is used for outpatients and the device is mobile and designed to be worn on the patients skin or on a belt. The power supply may contain a digital function generator and in some cases a digital amplifier.

The function generator, which may comprise a programmable 0-30 MHz arbitrary waveform generator, sends pulses of power to the transducer. In some cases, the function generator can be swept and used to generate a range of frequencies of ultrasound from the transducer. The duty cycle may be varied from continuous to 0%. The number of bursts may be varied from 1 to more than a 1,000. The intensity of the bursts may vary from about a 0.001-volt peak-to-peak intensity to about 100 volts peak-to-peak. The average power to the transducer may vary from about 0.001 Watts or even lower to about 100 Watts. The power is adjusted to achieve the desired drug delivery rate. Preferably the power is modulated over time so as to deliver physiologically acceptable concentrations of the drug.

As would be understood by one skilled in the art, the term “drug” is meant to include all manner of therapeutic compositions for which transdermal delivery could be employed as a method of treatment. Such compositions may include, but are not limited to, amino acids, peptides and proteins, nucleic acids, DNA or RNA, anti-fungal agents, antibiotics, hormones, vitamins, anti-coagulation agents, antivirals, anti-inflammatories, local anesthetics, radioactive agents and combinations thereof.

Preferably, the drug is an analgesic such as fentanyl, dynorphins, deltorphins or endorphin analogs, kappa antagonists or prostaglandin synthetase inhibitors.

Most preferably, the therapeutic agent is a molecule that is generally difficult to deliver via the oral route. Most preferred are therapeutic agents that contain amino acids, preferably peptides and proteins. Useful such materials include but are not limited to insulin, growth hormone, leutiniizing hormone, leutinizing releasing hormone, leutinizing releasing hormone inhibitor, interferons, interleukins, erythropoeitin, granulocyte macrophage colony stimulating factor and many other proteins and peptides. A particularly preferred molecule is insulin for transdermal delivery.

Typical systemically active agents which may be delivered transdermally are therapeutic agents which are sufficiently potent such that they can be delivered through the skin or other membrane to the bloodstream in sufficient quantities to produce the desired therapeutic effect. In general, this includes therapeutic agents in all of the major therapeutic areas including, but not limited to, anti-infectives, such as antibiotics and antiviral agents, analgesics and analgesic combinations, anorexics, anthelmintics, antiarthritics, antiasthma agents, anticonvulsants, antidepressants, antidiabetic agents, antidiarrheals, antihistamines, anti-inflammatory agents, antimigraine preparations, antimotion sickness, antinauseants, antineoplastics, antiparkinsonism drugs, antipruritics, antipsychotics, antipyretics, antispasmodics, including gastrointestinal and urinary; anticholinergics, sympathomimetics, xanthine derrivatives, cardiovascular preparations including calcium channel blockers, beta-blockers, antiarrhythmics, antihypertensives, diuretics, vasodilators including general, coronary, peripheral and cerebral; central nervous system stimulants, cough and cold preparations, decongestants, diagnostics, hormones, hypnotics, immunosuppressives, muscle relaxants, parasympatholytics, parasympathomimetics, psychostimulants, sedatives and tranquilizers.

Other suitable therapeutics include, but are not limited to: antineoplastic agents, such as platinum compounds (e.g., spiroplatin, cisplatin, and carboplatin), methotrexate, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon .alpha.-2a, interferon .alpha.-2b, teniposide (VM-26), vinblastine sulfate (VLB), vincristine sulfate, bleomycin, bleomycin sulfate, methotrexate, adriamycin, and arabinosyl; blood products such as parenteral iron, hemin, hematoporphyrins and their derivatives; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents such as ketoconazole, nystatin, griseofulvin, flucytosine (5-fc), miconazole, amphotericin B, ricin, and .beta.-lactam antibiotics (e.g., sulfazecin); hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; vitamins such as cyanocobalamin neinoic acid, retinoids and derivatives such as retinol palmitate and alpha-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as acyclovir, amantadine azidothymidine (AZT or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium.

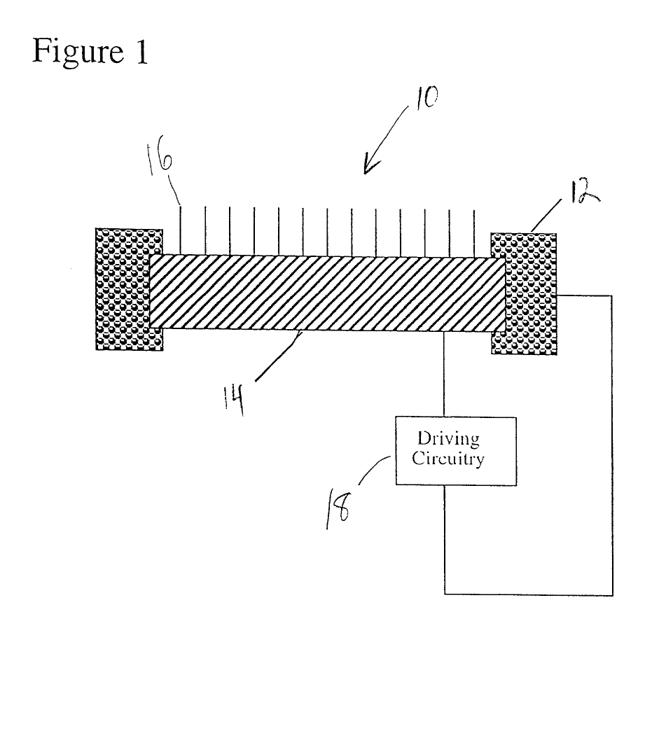

As depicted in FIG. 1, which shows a cross-sectional, schematic view of the device, the invention 10 generally comprises a drug reservoir 12, an acoustic transducer 14, an array of microneedles 16, and a driving circuitry 18. The microarray needles 16 themselves may be piezoelectric or vibrate from a piezoelectric material. Additionally and optionally, the microneedles 16 may also be electrically conducting and designed to impart an electric current in the tissue. The current direction in the needles may be adjusted so as to change the polarity of the electric discharge. This can be performed with a switch to allow the operator to choose (+) or (−) current for the treatment needles. For sampling biomolecules the needles 16 may be adjusted to the opposite charge of a given biomolecule.

Again referring to FIG. 1, the drug reservoir 12 may be positioned at the periphery of the transducer 14 or may also be incorporated into the transducer 14 face. Preferably a semi-permeable membrane (not shown) is positioned towards the skin with a reservoir of drug within the membrane or on the surface thereof. Accordingly, the reservoir 12 may take the form of a simple patch. The drug(s) may be contained within a patch in the form of a hydrogel material. The hydrogel may be made of materials as are well known in the art such as synthetic polymers including but not limited to simethicone, silica gel, silica rubber, polyvinylalcohol, polyethyleneglycol, polymethacrylate, polypropyleneglycol, copolymers and derivatives with and without cross-linking and other polymers such as alginic acid, pectins, albumin, collagen and other materials suitable for forming a gel to contain the desired drug into the form of a patch.

The drug reservoir 12 may also contain a variety of skin penetration enhancers such as surfactants, ionic and non-ionic. A penetration enhancer or permeation enhancer is an agent used to increase the permeability of the skin to a pharmacologically active agent to increase the rate at which the drug diffuses through the skin and enters the tissues and bloodstream. A chemical skin penetration enhancer increases skin permeability by reversibly damaging or by altering the physiochemical nature of the stratum corneum to reduce its diffusional barrier qualities.

According to technical and patent literature up to 1996, more than 275 different chemical compounds were found to be cited as skin penetration enhancers. Most of the compounds are generally recognized as safe ingredients that would often be considered inert by a formulator. Osborne D W, Henke J J, Pharmaceutical Technology, November 1997, pp 58-86. Examples of surfactant skin penetration enhancers could include tween, Pluronics, fatty acids, phospholipids, polyethyleneglycol, glycerol, propylene glycol, fluorosurfactants and other penetration enhancers as are well known in the art.

Examples of other types of penetration enhancers include: alcohols, such as ethanol and isopropanol; polyols, such as n-alkanols, limonene, terpenes, dioxolane, propylene glycol, ethylene glycol or other glycols. Penetration enhancers for the purpose of this invention may further be selected from the group consisting of: alcohols, polyols, sulfoxides, esters, ketones, amides, oleates, surfactants, alkanoic acids or lactam compounds. Other penetration enhancers such as alkanes, alkenes, alcohols, amides, amines, amine oxides, carboxylic acids, ethers, esters, halocarbons, ketones, and sulfoxides.

These penetration enhancers may be present primarily in either the oil-like phase of the emulsion or the hydroalcoholic phase. Non-limiting examples of additional penetration enhancers include C8±C22 fatty acids such as isostearic acid, octanoic acid, and oleic acid.

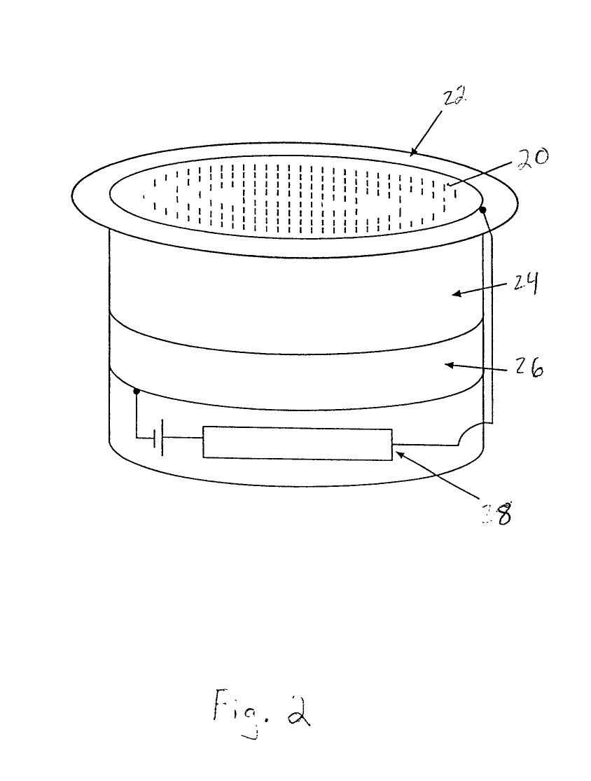

FIG. 2 shows a schematic side view of a preferred device of the invention. In this embodiment, a circular needle array 20 is surrounded by an annular flange 22 that is impregnated with a drug or drugs. Acoustic element 24 sits atop a backing material 26 with a selected acoustic impedance. When the sound is directed away from the backing material, an acoustically reflective material is generally selected, such as metal, e.g. aluminum foil, or an air backing. When the sound is to be transmitted through the backing, then an acoustically transparent material is selected, e.g. a polyvinyl material. Power source 28 provides electricity for the acoustic element 24 for the production of ultrasound waves, and, optionally, to the needle array 20 if the application of an electric field to the treatment area is desired.

The device is preferably small and relatively flat so as to be worn as a patch on the patient's skin. The device may also contain a receiver and transmitter (not shown). The transmitter may use a Bluetooth method of transmission and reception of data. A second device positioned elsewhere on the skin, e.g. the opposite side of the patient's chest may communicate with the first device. The second device may use a similar method (pore formation) followed by force, e.g. a negative pressure as applied by a vacuum to extract interstitial fluids. Preferably such a second device will provide a negative pressure to withdraw fluid from the pores. The second device preferably contains an integrated circuit for analyzing fluid and measuring the concentration of one or more biologically relevant molecules, e.g. glucose. The second device transmits signals over a Bluetooth or other network to the first device, which then receives this information. The first device has a program which controls the power supply and function generator to the transducer. The power is then modulated depending upon the input to achieve physiological control by adjusting the rate of delivery of the medication.

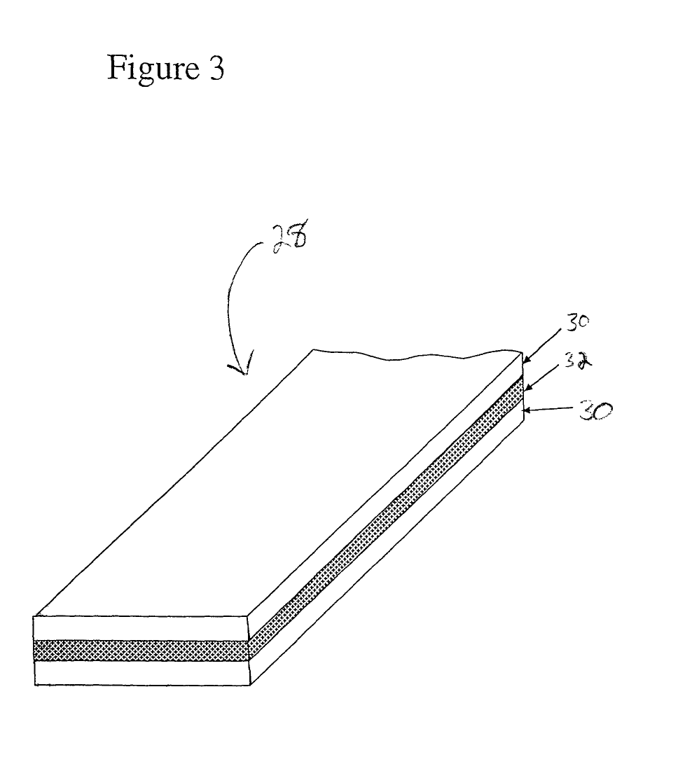

As seen in FIG. 3, the needle array may be formed through a “sandwich” type structure 28. The structure includes a sheet with three layers: electrically insulating layers 30 with an electrically conductive layer 32 in between the layers 30. The insulating layers 30 may be rather simple, such as a coating of resin. The conductive layer 32 is composed of fibers made from conductive materials, such as carbon (including carbon nanotubes), copper, stainless steel, titanium, or other composite materials. The thickness of the entire sheet is 50 microns to 10 millimeters, preferably 0.1 to 5 millimeters.

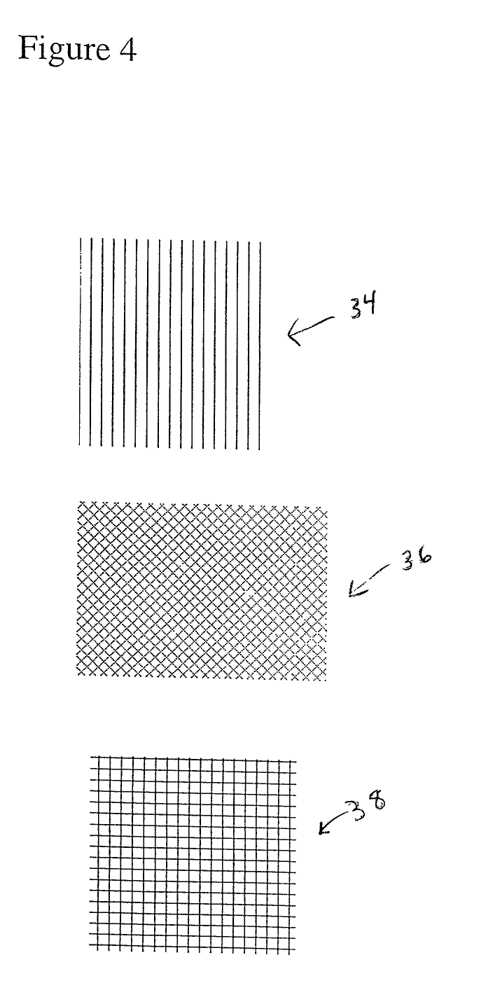

FIG. 4 shows in detail preferred components of the electrically conductive layer that may also form the microneedle array. Parallel stacked fibers 34 are arranged on a backing (not shown), with an electrically conductive coating used to fill the gap so that the entire arrangement is conductive. A second layer of intertwined fibers 36 is superimposed upon the stacked fibers 34. Finally, a third layer of gridded fibers 38 is added to the first two layers. Of course, other layouts or arrangements are also possible.

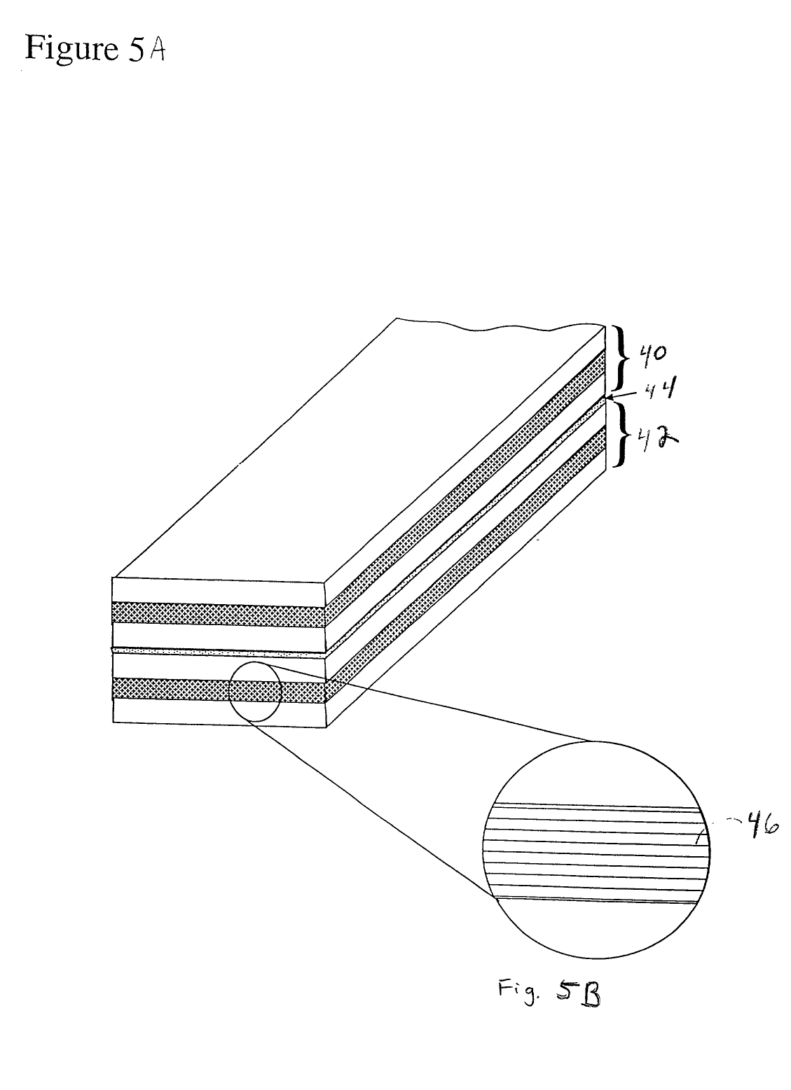

FIG. 5A shows electrically conducting layers sandwiched between insulating layers in a stacked arrangement. A first sheet of insulated, conductive fibers 40 and a second sheet of insulated, conductive fibers 42 are stacked with an adhesive layer 44. In magnified view FIG. 5B, parallel fibers 46 are shown in an orientation that would allow them to form a microneedle array if exposed as described below.

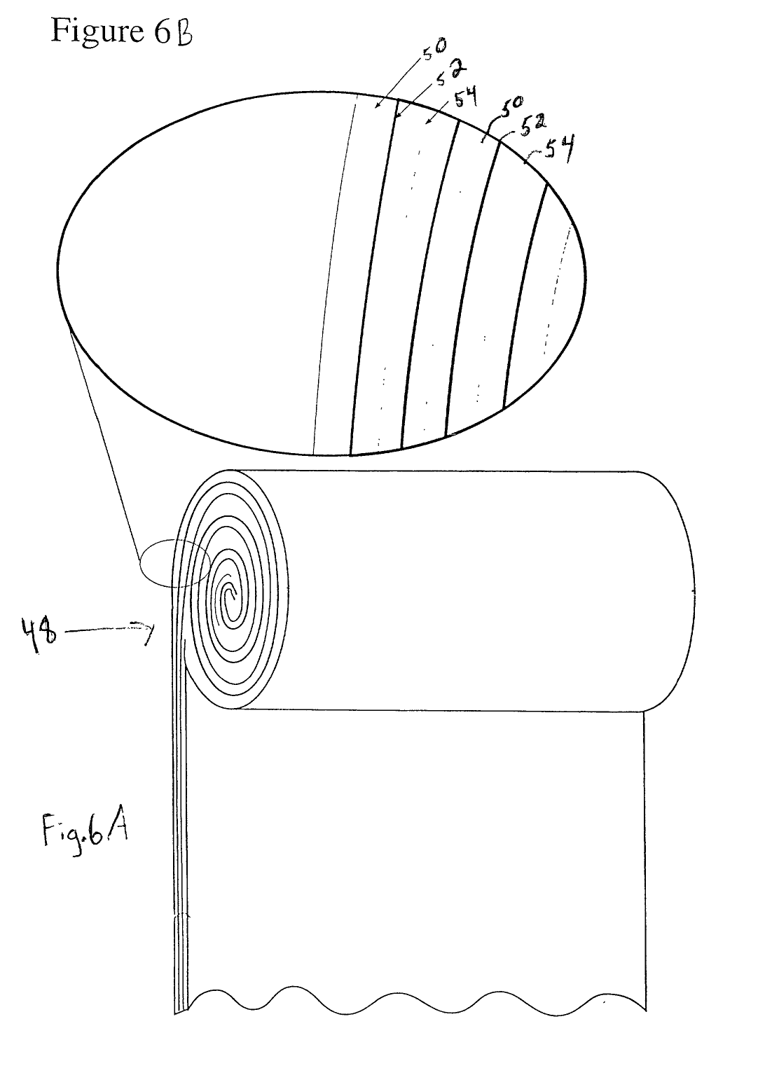

FIG. 6A shows how the sheets 40 and 42 of FIG. 5A can be rolled to create a cylinder 48 of concentric layers of conducting and insulating layers. FIG. 6B better shows in magnified detail the individual layers of the cylinder 48, which includes insulating layer 50, conductive layer 52, and adhesive layer 54.

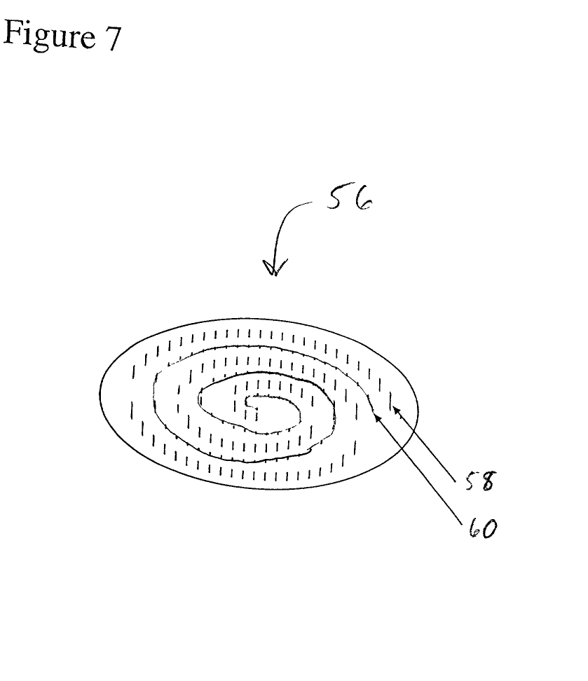

To make the microneedle array of the device, the concentric layers can be cut at intervals ranging from about 200 microns to about 5 cm. As illustrated in FIG. 7, top face 56 has microneedles 58 exposed by etching away the embedding:.material from the layer containing the microneedles 58 and insulating layer 60 to prepare the microarray of needles. For example, when epoxy resin is used as the embedding material, the embedding material may be removed by etching away the resin with hydrochloric acid. The resulting array of needles will then be applied to the skin such that the long axis of the needles is perpendicular to the skin.

Various materials may be used to construct the microneedles including metals such as titanium, steel, aluminum, copper, gold, platinum and alloys. Ceramic, silica and polymeric materials may also be employed, as well as carbon fibers. In the case of carbon, ceramic or polymeric materials, various dopants, including metal ions and other molecules may also be used to adjust the electrical potential of the microneedles to the desired characteristics.

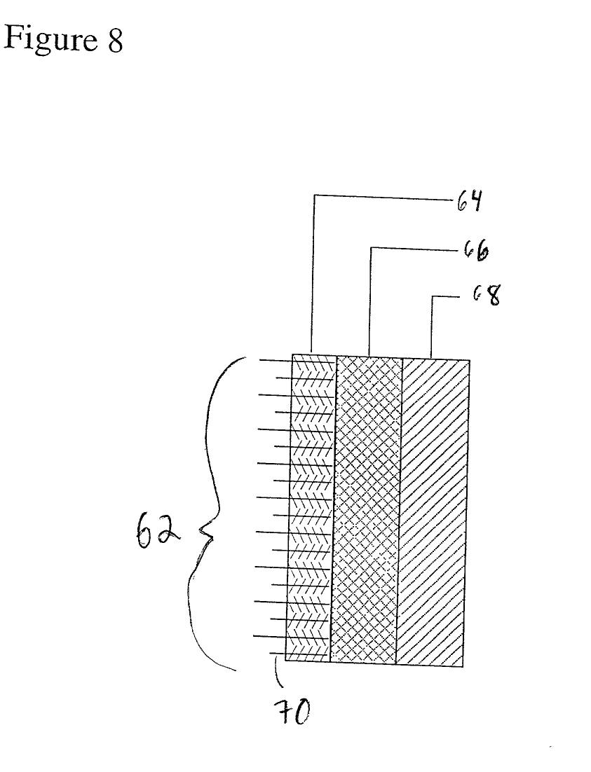

FIG. 8 shows a partial, cross-sectional view of the assembled components of the preferred device. The face 62 of the microarray of needles 64 is positioned on the top end of the transducer 66. Thus, the acoustic element in this case is positioned posterior to the microarray needles. When the transducer 66 fires it may cause the microarray needles to vibrate and/or respond electrically. The backing material 68 may include air or a housing, which in turn may contain the function generator and battery (not shown).

Most preferably the face 62, microneedles 70 and drug reservoir (not shown) are disposable and are contained together in the form of a patch. The acoustic elements of the transducer 66 may be reusable but the whole device may be provided as one unit and sold as a disposable.

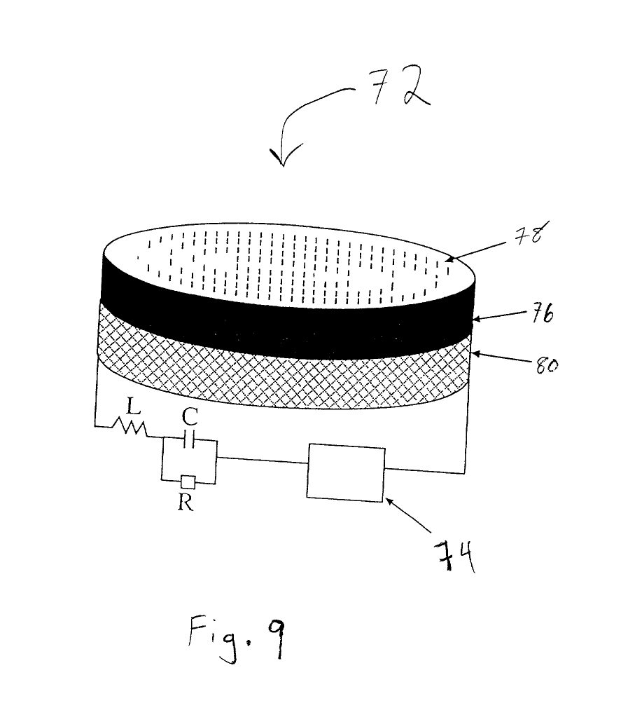

FIG. 9 shows a side schematic view of the completed microneedle array and transducer assembly 72, which may be worn, in the form of a patch. The driving circuit 74 functions to vary to supply of power to the transducer 76 and microarray of needles 78 housed atop the backing 80 to optimize the therapeutic result. A drug may be topically applied to the biological membrane of a patient before the microneedle array and transducer assembly 72 is worn on the same area. Preferably, a reservoir that is an integral part of the drug delivery assembly, similar to that shown in FIG. 1, provides the drug to the biological membrane surface.

### Prophetic Examples

1. A patient with pancreatic cancer has intractable pain. He is prescribed dynorphin to be delivered with the transdermal device. A patch containing a array of microneedles, a drug reservoir of dynorphin, and a transducer assembly is placed on the patient's chest. The patient is given a button to press when he wants another dose of pain medication. The patient depresses the button and it sends a signal via radiowaves over a Bluetooth network to the patch on his chest. The function generator is activated to deliver a burst of energy to the transducer and microneedle array.

2. A patient has poorly controlled diabetes. The patient is fitted with two devices. The first device comprises a microneedle array and transducer assembly with a backing that generates suction via vacuum pressure onto the faceplate and the skin. The device is activated to engage the transducer and electrically charge on the microneedles. A small quantity of about 100 microliters of interstitial fluid is withdrawn by the device and loaded into an analytical chamber. The chamber uses a glucose sensor to measure the concentration of glucose in the fluid. The information regarding glucose concentration is relayed via a radiosignal (the signal also could be relayed via ultrasound or infrared light) to the second device. The second device comprises another microneedle array and transducer assembly, in this case the assembly includes a drug reservoir filled with insulin. The signal is received from the first device by the second device and the power is adjusted to the second device to deliver the requisite dose of insulin.

3. A patient with pain is to be treated. The skin on the patient's chest is cleaned with an alcohol swab. A cylinder of compressed helium with a nozzle bearing a plurality of micronozzles is pressed against the skin and the gas lever is depressed. The gas expands and is expelled with high velocity against the patients skin producing a plurality of pores. Microbubbles of helium gas are entrapped within the pores in the patient's skin.

A patch bearing fentanyl is affixed to the patients skin. The back of the patch bears a transducer element. The patient is given a button and instructed to depress the button in response to pain. The patient depresses the button activating the ultrasound transducer. As the transducer is activated, in this case at 1.0 MHz, the microbubbles within the patient's skin resonate and increase acoustic streaming of fluid and drug across the skin. This causes the transdermal transport of fentanyl to increase rapidly. Of note, a basal level of transport can be attained through the patch alone and also by using low level intermittent pulsing of the ultrasound. Bolus delivery is achieved of larger quantities of drug as the energy to the transducer is increased.

4. This situation is the same as in Example 3, except that the patient is equipped with a pulse oximeter and respiratory meter. The patient receives a low level continuous infusion of drug from the pores/patch combination and low intensity ultrasound. The patient repeatedly depresses the button and receives multiple bolus doses of fentanyl. As the cumulative doses increase the patient becomes drowsy and his respiratory rate decreases. The pulse oximeter and respiratory meter detect the decrease in blood oxygen saturation and respirations. Signals indicating decreased blood oxygen and respirations are relayed to the device and ultrasound is stopped and the lockout interval (the time between doses that the patient receives from depressing the button) is increased.

5. An angioplasty catheter contains a central piezoelectric transducer as an array built into the distal end of the catheter.

The angioplasty balloon is covered with an array of microneedles, which lay flat while the balloon is depressed. The balloon is filled with a degassed solution containing Botulinum toxin. The Balloon is inflated using hydrostatic pressure to 6 atmospheres and the ultrasound transducer is activated. The microneedles enter the endoluminal surface of the blood vessel as the balloon is inflated. As the ultrasound is activated, this increases the driving force to deliver the drug into the blood vessel wall.

The botulinum toxin paralyzes the smooth muscle cells thereby decreasing vascular spasm and decreasing the propensity for smooth muscle proliferation. As one skilled in the art would recognize, the above example could be repeated with a variety of anti-thrombotic medications and drugs known to inhibit fibrointimal hyperplasia.

The disclosures of each patent and publication cited in this specification are hereby incorporated herein by reference, in their entirety.

As would be understood by those skilled in the art, any number of functional equivalents may exist in lieu of the preferred embodiments and methods described above. Thus, as will be apparent to those skilled in the art, changes in the details, steps and materials that have been described may be within the principles and scope of the invention illustrated herein and defined in the appended claims. Therefore, while the present invention has been shown and described in what is believed to be the most practical and preferred embodiments, it is recognized that departures can be made therefrom within the scope of the invention, which is therefore not to be limited to the details disclosed herein but is to be accorded the full scope of the claims so as to embrace any and all equivalent products and methods.

We claim:

1. A drug delivery method comprising the steps of:
a. creating pores across a biological membrane,
b. applying a drug from a reservoir to the biological membrane, and
c. exerting a pressure upon the drug, thereby transferring the drug through the membrane.

2. The method of claim 1, wherein the pores are created by an array of microneedles.

3. The method of claim 1, wherein the pores are created by a stream of compressed gas.

4. The method of claim 1, wherein the pores are created by burning holes in the membrane.

5. The method of claim 1, wherein the pressure generated in step c is in the form of ultrasound.

6. The method of claim 1, wherein the pressure generated in step c is in the form of static pressure.

7. The method of claim 6, wherein the static pressure is generated through expansion of a gas.

8. The method of claim 7, wherein said gas expansion is controlled by a heating element.

9. The method of claim 6, wherein said static pressure is generated by an actuator acting upon the drug from the reservoir to generate hydrostatic pressure.

10. The method of claim 1, wherein the reservoir is in the form of a patch.

11. A method for sampling biological fluids, comprising the steps of:
a. applying microneedles to tissue of a patient; and
b. applying energy adapted to produce a negative pressure upon the tissue from a power source such that the biological fluids are withdrawn from the tissue.

12. A method for sampling biological fluids, comprising the steps of:
a. making pores in the surface of a biological membrane; and
b. applying a negative pressure to a surface of the biological membrane.

13. The method of claim 12, wherein the negative pressure is produced by electricity.

14. The method of claim 12, wherein the negative pressure is produced by ultrasound.

15. The method of claim 12, wherein the negative pressure is produced by a combination of ultrasound and electricity.

16. The method of claim 12, further comprising measuring a concentration of a biomolecule within the biological fluids.

17. The method of claim 16, wherein the biomolecule is selected from the group consisting of an electrolyte, a hormone, a peptide, or a protein.

18. The method of claim 17, wherein the biomolecule comprises sodium, potassium, a hydrogen ion, Hemoglobin A-1C, an interferon, an interleukin, insulin, a growth factor, herceptin, or an antibody.

19. The method of claim 12, further comprising the steps of:
a. measuring a concentration of biological indicators in the biological fluids with a first device, and
b. relaying information about the concentration of said indicators from the first device to a second device, wherein concentration inputs are used to control energy output of the second device.

20. The method of claim 19, wherein the first and second devices are of unitary construction.

21. A method of in vivo drug delivery into targeted cells, said method comprising application of a pulsed electric field and ultrasonic waves substantially contemporaneously with administration of a drug, such that the drug is introduced into the targeted cells.

22. The method of claim 21, wherein the ultrasonic waves are 50 kHz to 10 MHz in frequency.

23. The method of claim 21, wherein the ultrasonic waves are 0.5 MHz to 2 MHz in frequency.

24. Live cells treated by the method of claim 21.

25. A controllable programmable drug delivery device, comprising an array of microneedles, an energy source, and means for controlling delivery of power to the device.

26. The device of claim 25, wherein said means includes a wireless network.

27. The device of claim 25, wherein said means includes a means for measureing a concentration of a biological molecule.

28. An all-in-one drug delivery device for administering drugs or withdrawing biological fluids through a biological membrane, comprising
a. a source of ultrasonic energy adapted to act on the membrane,
b. a source of electricity,
c. at least two microneedles, the microneedles being connected to said source of electricity, and
d. a drug reservoir in fluid connection with said microneedles.

29. The device of claim 28, wherein the source of electricity is a piezoelectric material.

30. The device of claim 29, wherein the source of ultrasonic energy is adapted to act upon the piezoelectric material.

31. The device of claim 28, wherein the drug delivery device is portable.

32. A drug delivery device for application to a biological membrane, comprising
a microneedle array;
a drug reservoir in fluid connection with said microneedle array; and
an ultrasonic transducer configured to contact the biological membrane with sound waves.
